(19) United States
(12) Patent Application Publication
(10) Pub. No.: US 2024/0252801 A1
(43) Pub. Date: Aug. 1, 2024

(54) Catheter Apparatus and Associated Methods of Use

(71) Applicant: ACCUMEDICAL BEIJING LTD., Beijing (CN)

(72) Inventors: Evan Michael Cohen, Costa Mesa, CA (US); Tai Dac Tieu, Fountain Valley, CA (US)

(73) Assignee: ACCUMEDICAL BEIJING LTD., Beijing (CN)

(21) Appl. No.: 18/104,616

(22) Filed: Feb. 1, 2023

(51) Int. Cl. A61M 39/06; A61M 25/00

(52) U.S. Cl. CPC A61M 39/06; A61M 25/0026; A61M 2039/062

(57) ABSTRACT

An improved catheter apparatus is disclosed for use with a hemostasis valve having a compression seal for temporarily creating a reduced inner diameter within a valve lumen of the valve. In at least one embodiment, the apparatus provides an elongate catheter having at least one protrusion radially extending outwardly from an outer surface of the catheter, the at least one protrusion having an outer diameter that is less than a neutral inner diameter of the valve lumen but greater than the reduced inner diameter of the valve lumen. During use of the apparatus, with the catheter positioned a distance within the valve lumen and the compression seal subsequently creating the reduced inner diameter within the valve lumen, the at least one protrusion contacts the compression seal and prevents the catheter from sliding out of the valve.

### RELATED APPLICATIONS

Not applicable.

### BACKGROUND

The subject of this patent application relates generally to catheters, and more particularly to an improved catheter apparatus capable of creating a sufficient seal with a hemostasis valve while maintaining both the structural integrity of the catheter as well as the position of the catheter within a sheath that is engaged with the hemostasis valve.

Applicant hereby incorporates herein by reference any and all patents and published patent applications cited or referred to in this application.

By way of background, hemostasis valves are routinely used in neurovascular procedures for continuous irrigation of tubes, catheters, dilators and the like (hereinafter collectively referred to as “catheters” for simplicity purposes) so as to minimize blood loss and decrease the risk of thromboembolism. Thus, hemostasis valves are employed for leak-proof introduction of catheters into the circulatory system of a patient or elsewhere in the body of the patient. Typically, a relatively larger diameter sheath, catheter or dilator (hereinafter collectively referred to as a “sheath” for simplicity purposes) is connected to a second end of the hemostasis valve, and the catheter is inserted into the first end and through the sheath to the desired location in the patient. After the catheter is in place, the valve is closed to prevent blood from escaping from the body of the patient via the sheath and/or catheter.

Conventional hemostasis valves—often referred to as rotating hemostasis valves (“RHV”)—have a rotating compression seal at the first end, which is rotated open or closed each time a catheter is introduced or extracted. In a bit more detail, the compression seal typically comprises a deformable gasket positioned within a tapered opening, such that when the seal is rotated into the closed position, the gasket is deformed until the gasket clamps down on the catheter. Each time the catheter is adjusted, the seal must first be rotated into the open position to allow for movement of the catheter, and then subsequently rotated back into the closed position to reestablish the seal after the adjustment. During the time that the valve is open, blood and other fluids leak from the patient. Similarly, if the seal is not closed tightly enough around the catheter during manipulation, blood and other fluids may leak from the patient via the sheath and/or catheter, which can result in stagnation of blood within the sheath or catheter and become a potential source of emboli during a procedure. Additionally, if the seal is not closed tightly enough around the catheter, forward movement of the sheath and catheter within the patient—such as during initial insertion or subsequent movement of the sheath—could unintentionally cause the catheter to be pushed a distance back through the sheath and out the first end of the hemostasis valve. On the other hand, when the seal is closed too tightly around the catheter, the sheath and/or catheter could be damaged.

Thus, there remains a need for an improved catheter that is capable of creating a sufficient seal with a hemostasis valve while maintaining both the structural integrity of the catheter as well as the position of the catheter within the sheath that is engaged with the hemostasis valve. Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing, in at least one embodiment, an improved catheter apparatus for use with a hemostasis valve, the hemostasis valve providing a valve body having a first end and an opposing second end with a valve lumen extending therebetween, the hemostasis valve further providing an elongate sheath engaged with the second end of the valve body and in fluid communication with the valve lumen, and a compression seal positioned substantially proximal to the first end of the valve body and configured for temporarily creating a reduced inner diameter within the valve lumen. In at least one such embodiment, the apparatus provides an elongate catheter having a first end and an opposing second end, an outer surface of the catheter having an outer diameter that is less than a neutral inner diameter of the valve lumen, thereby allowing the catheter to be slidably inserted a distance into the valve lumen. An at least one protrusion radially extends outwardly from the outer surface of the catheter, the at least one protrusion having an outer diameter that is less than the neutral inner diameter of the valve lumen but greater than the reduced inner diameter of the valve lumen. During use of the apparatus, with the catheter positioned a distance within the valve lumen and the compression seal subsequently creating the reduced inner diameter within the valve lumen, the at least one protrusion contacts the compression seal and prevents the catheter from sliding toward the first end of the valve body.

In at least one further embodiment, a combination catheter and hemostasis valve system provides a hemostasis valve comprising a valve body having a first end and an opposing second end with a valve lumen extending therebetween, the valve lumen having a neutral inner diameter; an elongate sheath engaged with the second end of the valve body and in fluid communication with the valve lumen; and a compression seal positioned substantially proximal to the first end of the valve body and configured for temporarily creating a reduced inner diameter within the valve lumen. Accordingly, the hemostasis valve is configured for selectively moving between one of a closed state—wherein the compression seal radially collapses so as to create the reduced inner diameter within the valve lumen—and an open state—wherein the compression seal radially moves outwardly so as to no longer create the reduced inner diameter within the valve lumen. The system further provides a catheter apparatus comprising an elongate catheter having a first end and an opposing second end, an outer surface of the catheter having an outer diameter that is less than a neutral inner diameter of the valve lumen, thereby allowing the catheter to be slidably inserted a distance into the valve lumen. An at least one protrusion radially extends outwardly from the outer surface of the catheter, the at least one protrusion having an outer diameter that is less than the neutral inner diameter of the valve lumen but greater than the reduced inner diameter of the valve lumen. During use of the apparatus, with the catheter positioned a distance within the valve lumen and the hemostasis valve subsequently moved into the closed state, the at least one protrusion contacts the compression seal and prevents the catheter from sliding toward the first end of the valve body.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIGS. 1 and 2 are partial side elevational views of an exemplary catheter apparatus, in accordance with at least one embodiment;

FIG. 3 is a partial side elevational view of a further exemplary catheter apparatus, in accordance with at least one embodiment;

FIG. 4 is a partial side elevational view of a still further exemplary catheter apparatus, in accordance with at least one embodiment;

FIGS. 5 and 6 are cross-sectional views of an exemplary hemostasis valve, in accordance with at least one embodiment; and

FIG. 7 is a partial cross-sectional view of the exemplary catheter apparatus of FIGS. 1 and 2 positioned within the exemplary hemostasis valve of FIGS. 4 and 5, in accordance with at least one embodiment.

The above-described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

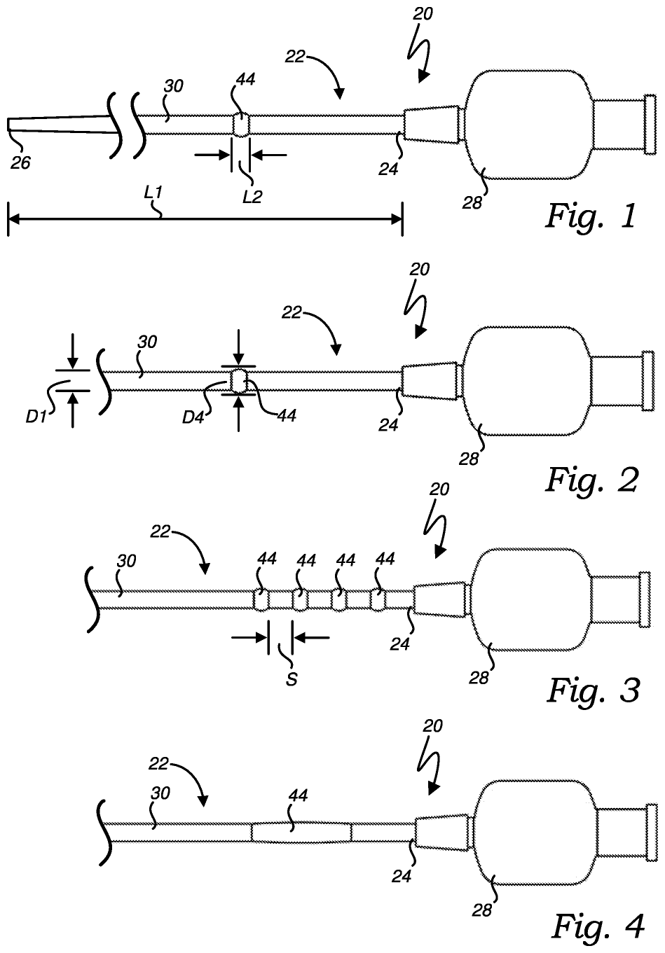

Turning now to FIG. 1, there is shown a partial side elevational view of an exemplary embodiment of an improved catheter apparatus 20. In at least one embodiment, the apparatus 20 provides an elongate catheter 22 having a first end 24 and an opposing second end 26, along with a hub 28 engaged with the first end 24 of the catheter 22. In at least one embodiment, the catheter 22 is constructed out of one or more of polyether, polyester, polyimide, nylon, polyurethane, polyetherimide, polyetheretherketone, polyamide, silicone, polyether block amide and polyethylene. However, in further embodiments, the catheter 22 may be constructed out of any other materials (or combinations of materials) now known or later developed—dependent at least in part on the specific context in which the apparatus 20 is to be utilized—so long as the apparatus 20 is able to substantially carry out the functionality described herein. At the outset, it should be noted that the catheter 22 and hub 28 depicted in the drawings are merely exemplary and are shown for illustrative purposes. Accordingly, in further embodiments, each of the catheter 22 and hub 28 may take on any other sizes, shapes and/or dimensions now known or later developed (including having a plurality of lumens rather than the single lumen depicted in the drawings)—dependent at least in part on the specific context in which the apparatus 20 is to be utilized—so long as the apparatus 20 is able to substantially carry out the functionality described herein. Additionally, in at least one alternate embodiment, the hub 28 may be omitted. It is also once again noted that the term “catheter” as used herein is intended to generally include any and all types of tubes, catheters, dilators and the like which might be used in endovascular procedures, such as neurovascular, peripheral and cardio procedures for example.

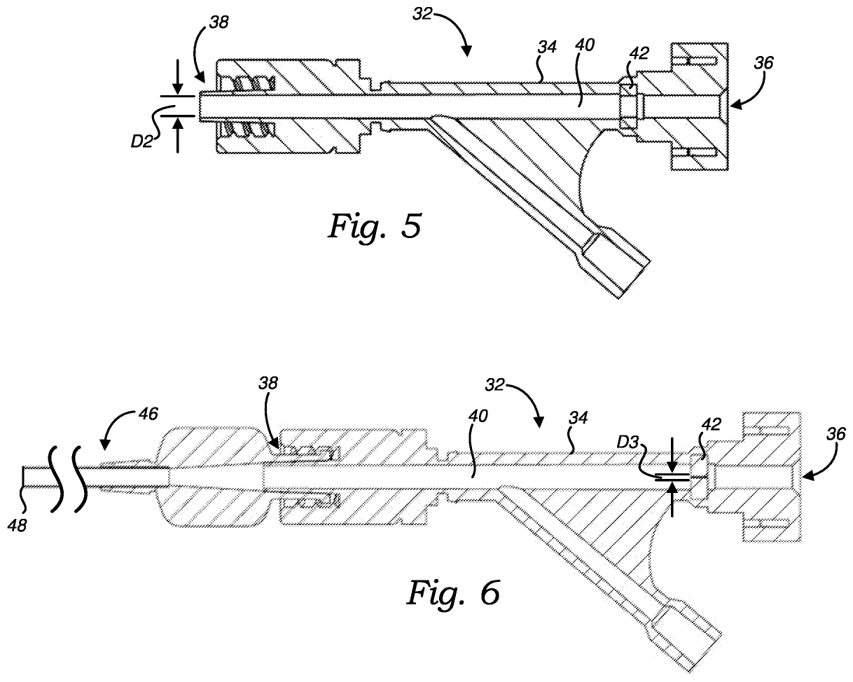

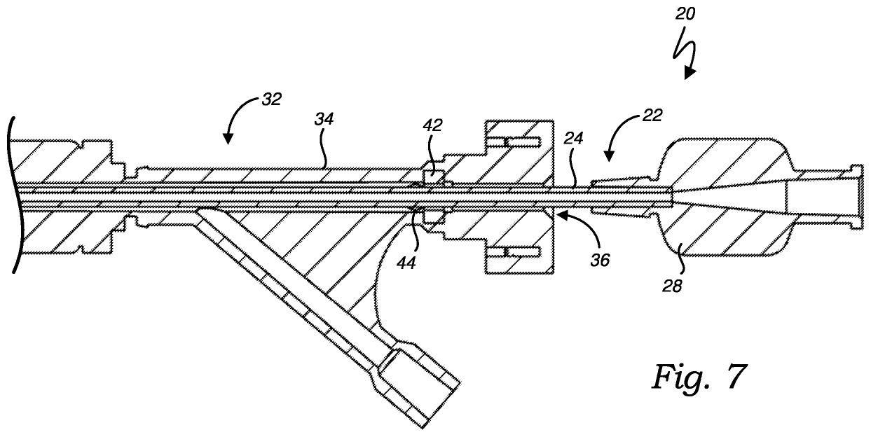

In at least one embodiment, an outer surface 30 of the catheter 22 has an outer diameter D1 (FIG. 2) that is sized for allowing the catheter 22 to be slidably inserted a distance into a hemostasis valve 32, as illustrated in FIG. 7. In at least one embodiment, the catheter 22 has an outer diameter D1 of about 1 French to about 34 French—dependent at least in part on the specific context in which the apparatus 20 is to be utilized; however, in further embodiments, the catheter 22 may have an outer diameter D1 of less than 1 French or more than 34 French. In at least one embodiment, the hemostasis valve 32 provides a valve body 34 having a first end 36 and an opposing second end 38 with a valve lumen 40 extending therebetween, the valve lumen 40 having a neutral inner diameter D2 (FIG. 5) that is relatively greater than the outer diameter D1 of the catheter 22, so as to slidably receive a length of the catheter 22 therethrough. Additionally, in at least one embodiment, as illustrated in FIG. 6, the second end 38 of the valve body 34 is engageable with an elongate sheath 46 (also sized for slidably receiving a length of the catheter 22 therethrough) in fluid communication with the valve lumen 40 and having a length that is relatively shorter than a length L1 of the catheter 22, such that the second end 26 of the catheter 22 may extend a distance beyond a terminal end 48 of the sheath 46 during use of the apparatus 20. The first end 36 of the valve body 34 is sized and configured for removably receiving the second end 26 of the catheter 22 as the catheter 22 is inserted into hemostasis valve 32, and further configured for subsequent frictional engagement with the outer surface 30 of the catheter 22 substantially proximal to the first end 24 (after the catheter 22 has been inserted a desired distance into the hemostasis valve 32). In at least one embodiment, the valve body 34 further provides a compression seal 42 positioned substantially proximal to the first end 36 of the valve body 34 and configured for radially collapsing or otherwise moving inwardly within the valve lumen 40 so as to clamp down on the catheter 22. Thus, the hemostasis valve 32 is configured for selectively moving between one of a closed state (FIG. 6)—wherein the compression seal 42 is radially moved inwardly so as to clamp down on the catheter 22—and an open state (FIG. 5)—wherein the compression seal 42 is radially moved outwardly so as to no longer frictionally engage the catheter 22. In other words, when the hemostasis valve 32 is in the closed state, the compression seal 42 temporarily creates a reduced inner diameter D3 within a portion of the valve lumen 40 that is less than the outer diameter D1 of the catheter 22. In at least one embodiment, as illustrated in FIG. 5, when the hemostasis valve 32 is in the open state, the compression seal 42 has an inner diameter that is substantially equal to the neutral inner diameter D2 of the valve lumen 40. It should be noted that the hemostasis valve 32 depicted in the drawings is merely exemplary and is shown for illustrative purposes. Accordingly, in further embodiments, the hemostasis valve 32 may take on any other sizes, shapes, dimensions, and/or configurations now known or later developed.

Referring again to FIGS. 1 and 2, in at least one embodiment, the outer surface 30 of the catheter 22 provides an at least one protrusion 44 radially extending outwardly from the outer surface 30 of the catheter 22, such that the at least one protrusion has an outer diameter D4 that is greater than the outer diameter D1 of the catheter 22. In at least one embodiment, the at least one protrusion 44 further extends around an entire circumference of the outer surface 30 of the catheter 22. In at least one alternate embodiment, the at least one protrusion 44 does not extend around the entire circumference of the outer surface 30 of the catheter 22. In at least one embodiment, the at least one protrusion 44 has an outer diameter D4 (FIG. 2) that is less than the neutral inner diameter D2 of the valve lumen 40 but greater than the reduced inner diameter D3 of the valve lumen 40 when the hemostasis valve 32 is in the closed state. As such, when the hemostasis valve 32 is in the open state, the catheter 22 is capable of freely sliding within the valve lumen 40; and when the hemostasis valve 32 is in the closed state, the at least one protrusion 44 of the catheter 22 prevents the catheter 22 from sliding toward the first end 36 of the valve body 34 (i.e., traveling backward) upon said at least one protrusion 44 coming into contact with the compression seal 42, as illustrated in FIG. 7 (i.e., the compression seal 42 traps the at least one protrusion 44 within the valve lumen 40). Additionally, upon the hemostasis valve 32 moving into the closed state, should the at least one protrusion 44 be positioned substantially in line with the compression seal 42 as the compression seal 42 radially collapses within the valve lumen 40, said at least one protrusion 44 is capable of increasing the structural integrity of the catheter 22 so as to better prevent the catheter 22 from becoming crushed or otherwise damaged by the compression seal 42.

In at least one embodiment, the at least one protrusion 44 is constructed out of one or more of polyether, polyester, polyimide, nylon, polyurethane, polyetherimide, polycarbonate, polyetheretherketone, polyamide, silicone, polyethylene, acrylic, acrylonitrile butadiene styrene, methylmethacrylate butadiene styrene, polypropylene, polyether block amide (“PEBA”), or any common polymer or combination resulting in a block copolymer. In at least one further embodiment, the at least one protrusion 44 is constructed out of one or more of stainless steel, nitinol, cobalt chromium, platinum-iridium, or other metals or alloys. In at least one further embodiment, the at least one protrusion 44 is constructed out of a tacky, high-friction material so as to increase the friction between the at least one protrusion 44 and the compression seal 42 when the hemostasis valve 32 is in the closed state. In at least one further embodiment, the at least one protrusion 44 is constructed out of a semi-compliant polymer so as to cooperate with the compression seal 42 to form a seal therebetween when the hemostasis valve 32 is in the closed state. In at least one further embodiment, the at least one protrusion 44 is constructed out of one or more radiopaque materials to facilitate positioning of the catheter 22 within the valve lumen 40. In at least one such embodiment, the at least one protrusion 44 is constructed out of a tungsten-loaded polymer. In further embodiments, the at least one protrusion 44 may be constructed out of platinum, chromium, cobalt, tantalum, nitinol, gold, silver, bismuth subcarbonate, barium sulfate, bismuth oxychloride, bismuth trioxide, stainless steel or alloys thereof, or any other radiopaque material (or combination of materials) now known or later developed. In still further embodiments, the at least one protrusion 44 may be constructed out of any other materials (or combinations of materials) now known or later developed—dependent at least in part on the specific context in which the apparatus 20 is to be utilized—so long as the apparatus 20 is able to substantially carry out the functionality described herein.

In at least one embodiment, the at least one protrusion 44 has an outer diameter D4 of about 100.1% to about 500% of the outer diameter D1 of the catheter 22—dependent at least in part on the neutral inner diameter D2 of the valve lumen 40 (given that the outer diameter D4 of the at least one protrusion 44 must be less than the neutral inner diameter D2 of the valve lumen 40, as discussed above). In further embodiments, the at least one protrusion 44 may have an outer diameter D4 that is greater than 500% of the outer diameter D1 of the catheter 22—again, dependent at least in part on the neutral inner diameter D2 of the valve lumen 40. In at least one embodiment, the at least one protrusion 44 has a length L2 of about 0.001 inch to about 20 inches; however, in further embodiments, the at least one protrusion 44 may have a length L2 of less than 0.001 inch or more than 20 inches. Thus, in further embodiments (as illustrated in the non-limiting examples of FIGS. 3 and 4), the at least one protrusion 44 may take on any other sizes, shapes, quantities, dimensions and/or relative positions now known or later developed—dependent at least in part on the specific context in which the apparatus 20 is to be utilized—so long as the apparatus 20 is able to substantially carry out the functionality described herein.

In that regard, in at least one embodiment, the position of the at least one protrusion 44 relative to the first end 24 of the catheter 22 is configured to indicate a distance by which the opposing second end 26 of the catheter 22 extends from the terminal end 48 of the sheath 46 engaged with the hemostasis valve 32 when said protrusion 44 is positioned substantially in line with the compression seal 42. Given that the terminal end 48 of the sheath 46 (along with the second end 26 of the catheter 22) will be positioned within the patient (and therefore not visible to the medical service provider operating the apparatus 20) during use of the apparatus 20, providing such a visual indicator near the first end 24 of the catheter 22 allows the medical service provider to determine how far the second end 26 of the catheter 22 extends beyond the terminal end 48 of the sheath 46. Furthermore, given that the at least one protrusion 44 may be frictionally engaged by the compression seal 42 in at least one embodiment (as discussed above), the medical service provider is essentially able to set the distance by which the opposing second end 26 of the catheter 22 extends from the terminal end 48 of the sheath 46 prior to actually inserting the apparatus 20 into the patient, by positioning the at least one protrusion 44 relative to the compression seal 42 as desired, then moving the hemostasis valve 32 into the closed state. In at least one such embodiment, as illustrated in FIG. 3, the outer surface 30 of the catheter 22 provides a plurality of spaced apart protrusions 44 along a length of the catheter 22, with each of the protrusions 44 spaced a distance S apart from one another (with said spacing being either uniform or varying). In such embodiments, each of the protrusions 44 is positioned for indicating a different distance by which the opposing second end 26 of the catheter 22 extends from the terminal end 48 of the sheath 46 when the corresponding protrusion 44 is positioned substantially in line with the compression seal 42. In at least one embodiment, the distance S between each of the protrusions 44 is about 0.001 inch to about 20 inches; however, in further embodiments, the distance S between each of the protrusions 44 may be less than 0.001 inch or more than 20 inches. In at least one embodiment, where the outer surface 30 of the catheter 22 provides a plurality of spaced apart protrusions 44, each of the protrusions 44 are of substantially similar sizes, shapes, dimensions, materials of construction and relative spacing. In at least one alternate embodiment, one or more of the protrusions 44 is of a different size, shape, dimensions, materials of construction and/or relative spacing as compared to one or more of the other protrusions 44.

Aspects of the present specification may also be described as the following embodiments:

1. A catheter apparatus for use with a hemostasis valve, the hemostasis valve providing a valve body having a first end and an opposing second end with a valve lumen extending therebetween, the hemostasis valve further providing an elongate sheath engaged with the second end and in fluid communication with the valve lumen, and a compression seal positioned substantially proximal to the first end and configured for temporarily creating a reduced inner diameter within the valve lumen, the apparatus comprising: an elongate catheter having a first end and an opposing second end, an outer surface of the catheter having an outer diameter that is less than a neutral inner diameter of the valve lumen, thereby allowing the catheter to be slidably inserted a distance into the valve lumen; and an at least one protrusion radially extending outwardly from the outer surface of the catheter, substantially proximal to the first end of the catheter, the at least one protrusion having an outer diameter that is less than the neutral inner diameter of the valve lumen but greater than the reduced inner diameter of the valve lumen; whereby, with the catheter positioned a distance within the valve lumen and the compression seal subsequently creating the reduced inner diameter within the valve lumen, the at least one protrusion contacts the compression seal and prevents the catheter from sliding toward the first end of the valve body.

2. The catheter apparatus according to embodiment 1, further comprising a hub engaged with the first end of the catheter.

3. The catheter apparatus according to embodiments 1-2, wherein the catheter is constructed out of one or more of polyether, polyester, polyimide, nylon, polyurethane, polyetherimide, polyetheretherketone, polyamide, silicone, polyether block amide and polyethylene.

4. The catheter apparatus according to embodiments 1-3, wherein the catheter has an outer diameter of about 1 French to about 34 French.

5. The catheter apparatus according to embodiments 1-4, wherein the catheter has an outer diameter of less than 1 French or more than 34 French.

6. The catheter apparatus according to embodiments 1-5, wherein the catheter has a length that is greater than a length of the sheath, such that the second end of the catheter may extend a distance beyond a terminal end of the sheath during use of the apparatus.

7. The catheter apparatus according to embodiments 1-6, wherein the at least one protrusion extends around an entire circumference of the outer surface of the catheter.

8. The catheter apparatus according to embodiments 1-7, wherein the at least one protrusion extends around less than an entire circumference of the outer surface of the catheter.

9. The catheter apparatus according to embodiments 1-8, wherein the at least one protrusion is constructed out of one or more of polyether, polyester, polyimide, nylon, polyurethane, polyetherimide, polycarbonate, polyetheretherketone, polyamide, silicone, polyethylene, acrylic, acrylonitrile butadiene styrene, methylmethacrylate butadiene styrene, polypropylene, polyether block amide (“PEBA”), or any common polymer or combination resulting in a block copolymer.

10. The catheter apparatus according to embodiments 1-9, wherein the at least one protrusion is constructed out of one or more of stainless steel, nitinol, cobalt chromium, platinum-iridium, or other metals or alloys.

11. The catheter apparatus according to embodiments 1-10, wherein the at least one protrusion is constructed out of a tacky, high-friction material so as to increase the friction between the at least one protrusion and the compression seal when the compression seal creates the reduced inner diameter within the valve lumen.

12. The catheter apparatus according to embodiments 1-11, wherein the at least one protrusion is constructed out of a semi-compliant polymer so as to cooperate with the compression seal to form a seal therebetween when the compression seal creates the reduced inner diameter within the valve lumen.

13. The catheter apparatus according to embodiments 1-12, wherein the at least one protrusion is constructed out of one or more radiopaque materials to facilitate positioning of the catheter within the valve lumen.

14. The catheter apparatus according to embodiments 1-13, wherein the at least one protrusion is constructed out of a tungsten-loaded polymer.

15. The catheter apparatus according to embodiments 1-14, wherein the at least one protrusion is constructed out of one or more of platinum, chromium, cobalt, tantalum, nitinol, gold, silver, bismuth subcarbonate, barium sulfate, bismuth oxychloride, bismuth trioxide, stainless steel or alloys thereof.

16. The catheter apparatus according to embodiments 1-15, wherein the at least one protrusion has an outer diameter of about 100.1% to about 500% of the outer diameter of the catheter.

17. The catheter apparatus according to embodiments 1-16, wherein the at least one protrusion has an outer diameter of greater than 500% of the outer diameter of the catheter.

18. The catheter apparatus according to embodiments 1-17, wherein the at least one protrusion has a length of about 0.001 inch to about 20 inches.

19. The catheter apparatus according to embodiments 1-18, wherein the at least one protrusion has a length of less than 0.001 inch or more than 20 inches.

20. The catheter apparatus according to embodiments 1-19, wherein the position of the at least one protrusion relative to the first end of the catheter indicates a corresponding distance by which the opposing second end of the catheter extends from the terminal end of the sheath when said protrusion is positioned substantially in line with the compression seal.

21. The catheter apparatus according to embodiments 1-20, further comprising a plurality of spaced apart protrusions along a length of the catheter, each of the protrusions positioned relative to the first end of the catheter so as to indicate a corresponding distance by which the opposing second end of the catheter extends from the terminal end of the sheath when said protrusion is positioned substantially in line with the compression seal.

22. The catheter apparatus according to embodiments 1-21, wherein the protrusions are spaced about 0.001 inch to about 20 inches apart from one another.

23. The catheter apparatus according to embodiments 1-22, wherein the protrusions are spaced less than 0.001 inch or more than 20 inches apart from one another.

24. A catheter apparatus for use with a hemostasis valve, the hemostasis valve providing a valve body having a first end and an opposing second end with a valve lumen extending therebetween, the hemostasis valve further providing an elongate sheath engaged with the second end, and a compression seal positioned substantially proximal to the first end and configured for temporarily creating a reduced inner diameter within the valve lumen, the apparatus comprising: an elongate catheter having a first end and an opposing second end; an outer surface of the catheter having an outer diameter that is less than a neutral inner diameter of the valve lumen, thereby allowing the catheter to be slidably inserted a distance into the valve lumen; the catheter having a length that is greater than a length of the sheath, such that the second end of the catheter may extend a distance beyond a terminal end of the sheath when the catheter is inserted a distance into the valve lumen; and an at least one protrusion radially extending outwardly from the outer surface of the catheter and having an outer diameter that is less than the neutral inner diameter of the valve lumen but greater than the reduced inner diameter of the valve lumen, the at least one protrusion positioned relative to the first end of the catheter so as to indicate a corresponding distance by which the opposing second end of the catheter extends from the terminal end of the sheath when said protrusion is positioned substantially in line with the compression seal; whereby, with the catheter positioned a distance within the valve lumen and the compression seal subsequently creating the reduced inner diameter within the valve lumen, the at least one protrusion contacts the compression seal and prevents the catheter from sliding toward the first end of the valve body.

25. A combination catheter and hemostasis valve system comprising: a hemostasis valve comprising: a valve body having a first end and an opposing second end with a valve lumen extending therebetween, the valve lumen having a neutral inner diameter; an elongate sheath engaged with the second end of the valve body and in fluid communication with the valve lumen; and a compression seal positioned substantially proximal to the first end of the valve body and configured for temporarily creating a reduced inner diameter within the valve lumen; the hemostasis valve configured for selectively moving between one of a closed state—wherein the compression seal radially collapses so as to create the reduced inner diameter within the valve lumen—and an open state—wherein the compression seal radially moves outwardly so as to no longer create the reduced inner diameter within the valve lumen; and a catheter apparatus comprising: an elongate catheter having a first end and an opposing second end, an outer surface of the catheter having an outer diameter that is less than the neutral inner diameter of the valve lumen, thereby allowing the catheter to be slidably inserted a distance into the valve lumen; and an at least one protrusion radially extending outwardly from the outer surface of the catheter, substantially proximal to the first end of the catheter, the at least one protrusion having an outer diameter that is less than the neutral inner diameter of the valve lumen but greater than the reduced inner diameter of the valve lumen; whereby, with the catheter positioned a distance within the valve lumen and the hemostasis valve subsequently moved into the closed state, the at least one protrusion contacts the compression seal and prevents the catheter from sliding toward the first end of the valve body.

26. The combination catheter and hemostasis valve system according to embodiment 25, further comprising a hub engaged with the first end of the catheter.

27. The combination catheter and hemostasis valve system according to embodiments 25-26, wherein the catheter is constructed out of one or more of polyether, polyester, polyimide, nylon, polyurethane, polyetherimide, polyetheretherketone, polyamide, silicone, polyether block amide and polyethylene.

28. The combination catheter and hemostasis valve system according to embodiments 25-27, wherein the catheter has an outer diameter of about 1 French to about 34 French.

29. The combination catheter and hemostasis valve system according to embodiments 25-28, wherein the catheter has an outer diameter of less than 1 French or more than 34 French.

30. The combination catheter and hemostasis valve system according to embodiments 25-29, wherein the catheter has a length that is greater than a length of the sheath, such that the second end of the catheter may extend a distance beyond a terminal end of the sheath during use of the apparatus.

31. The combination catheter and hemostasis valve system according to embodiments 25-30, wherein the at least one protrusion extends around an entire circumference of the outer surface of the catheter.

32. The combination catheter and hemostasis valve system according to embodiments 25-31, wherein the at least one protrusion extends around less than an entire circumference of the outer surface of the catheter.

33. The combination catheter and hemostasis valve system according to embodiments 25-32, wherein the at least one protrusion is constructed out of one or more of polyether, polyester, polyimide, nylon, polyurethane, polyetherimide, polycarbonate, polyetheretherketone, polyamide, silicone, polyethylene, acrylic, acrylonitrile butadiene styrene, methylmethacrylate butadiene styrene, polypropylene, polyether block amide (“PEBA”), or any common polymer or combination resulting in a block copolymer.

34. The combination catheter and hemostasis valve system according to embodiments 25-33, wherein the at least one protrusion is constructed out of one or more of stainless steel, nitinol, cobalt chromium, platinum-iridium, or other metals or alloys.

35. The combination catheter and hemostasis valve system according to embodiments 25-34, wherein the at least one protrusion is constructed out of a tacky, high-friction material so as to increase the friction between the at least one protrusion and the compression seal when the compression seal creates the reduced inner diameter within the valve lumen.

36. The combination catheter and hemostasis valve system according to embodiments 25-35, wherein the at least one protrusion is constructed out of a semi-compliant polymer so as to cooperate with the compression seal to form a seal therebetween when the compression seal creates the reduced inner diameter within the valve lumen.

37. The combination catheter and hemostasis valve system according to embodiments 25-36, wherein the at least one protrusion is constructed out of one or more radiopaque materials to facilitate positioning of the catheter within the valve lumen.

38. The combination catheter and hemostasis valve system according to embodiments 25-37, wherein the at least one protrusion is constructed out of a tungsten-loaded polymer.

39. The combination catheter and hemostasis valve system according to embodiments 25-38, wherein the at least one protrusion is constructed out of one or more of platinum, chromium, cobalt, tantalum, nitinol, gold, silver, bismuth subcarbonate, barium sulfate, bismuth oxychloride, bismuth trioxide, stainless steel or alloys thereof.

40. The combination catheter and hemostasis valve system according to embodiments 25-39, wherein the at least one protrusion has an outer diameter of about 100.1% to about 500% of the outer diameter of the catheter.

41. The combination catheter and hemostasis valve system according to embodiments 25-40, wherein the at least one protrusion has an outer diameter of greater than 500% of the outer diameter of the catheter.

42. The combination catheter and hemostasis valve system according to embodiments 25-41, wherein the at least one protrusion has a length of about 0.001 inch to about 20 inches.

43. The combination catheter and hemostasis valve system according to embodiments 25-42, wherein the at least one protrusion has a length of less than 0.001 inch or more than 20 inches.

44. The combination catheter and hemostasis valve system according to embodiments 25-43, wherein the position of the at least one protrusion relative to the first end of the catheter indicates a corresponding distance by which the opposing second end of the catheter extends from the terminal end of the sheath when said protrusion is positioned substantially in line with the compression seal.

45. The combination catheter and hemostasis valve system according to embodiments 25-44, further comprising a plurality of spaced apart protrusions along a length of the catheter, each of the protrusions positioned relative to the first end of the catheter so as to indicate a corresponding distance by which the opposing second end of the catheter extends from the terminal end of the sheath when said protrusion is positioned substantially in line with the compression seal.

46. The combination catheter and hemostasis valve system according to embodiments 25-45, wherein the protrusions are spaced about 0.001 inch to about 20 inches apart from one another.

47. The combination catheter and hemostasis valve system according to embodiments 25-46, wherein the protrusions are spaced less than 0.001 inch or more than 20 inches apart from one another.

In closing, regarding the exemplary embodiments of the present invention as shown and described herein, it will be appreciated that an improved catheter apparatus is disclosed and configured for creating a sufficient seal with a hemostasis valve while maintaining both the structural integrity of the catheter as well as the position of the catheter within a sheath that is engaged with the hemostasis valve. Because the principles of the invention may be practiced in a number of configurations beyond those shown and described, it is to be understood that the invention is not in any way limited by the exemplary embodiments, but is generally directed to an improved catheter apparatus and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the terms “about” and “approximately.” As used herein, the terms “about” and “approximately” mean that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that any methods disclosed herein, along with the order in which the respective elements of each method are performed, are purely exemplary. Depending on the implementation, they may be performed in any order or in parallel, unless indicated otherwise in the present disclosure.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A catheter apparatus for use with a hemostasis valve, the hemostasis valve providing a valve body having a first end and an opposing second end with a valve lumen extending therebetween, the hemostasis valve further providing an elongate sheath engaged with the second end and in fluid communication with the valve lumen, and a compression seal positioned substantially proximal to the first end and configured for temporarily creating a reduced inner diameter within the valve lumen, the apparatus comprising:
an elongate catheter having a first end and an opposing second end, an outer surface of the catheter having an outer diameter that is less than a neutral inner diameter of the valve lumen, thereby allowing the catheter to be slidably inserted a distance into the valve lumen; and
an at least one protrusion radially extending outwardly from the outer surface of the catheter, the at least one protrusion having an outer diameter that is less than the neutral inner diameter of the valve lumen but greater than the reduced inner diameter of the valve lumen;
whereby, with the catheter positioned a distance within the valve lumen and the compression seal subsequently creating the reduced inner diameter within the valve lumen, the at least one protrusion contacts the compression seal and prevents the catheter from sliding toward the first end of the valve body.

2. The catheter apparatus of claim 1, further comprising a hub engaged with the first end of the catheter.

3. The catheter apparatus of claim 1, wherein the catheter is constructed out of one or more of polyether, polyester, polyimide, nylon, polyurethane, polyetherimide, polyetheretherketone, polyamide, silicone, polyether block amide and polyethylene.

4. The catheter apparatus of claim 1, wherein the catheter has an outer diameter of about 1 French to about 34 French.

5. The catheter apparatus of claim 1, wherein the catheter has a length that is greater than a length of the sheath, such that the second end of the catheter may extend a distance beyond a terminal end of the sheath during use of the apparatus.

6. The catheter apparatus of claim 1, wherein the at least one protrusion extends around an entire circumference of the outer surface of the catheter.

7. The catheter apparatus of claim 1, wherein the at least one protrusion is constructed out of one or more of polyether, polyester, polyimide, nylon, polyurethane, polyetherimide, polycarbonate, polyetheretherketone, polyamide, silicone, polyethylene, acrylic, acrylonitrile butadiene styrene, methylmethacrylate butadiene styrene, polypropylene, polyether block amide (“PEBA”), or any common polymer or combination resulting in a block copolymer.

8. The catheter apparatus of claim 1, wherein the at least one protrusion is constructed out of one or more of stainless steel, nitinol, cobalt chromium, platinum-iridium, or other metals or alloys.

9. The catheter apparatus of claim 1, wherein the at least one protrusion is constructed out of a tacky, high-friction material so as to increase the friction between the at least one protrusion and the compression seal when the compression seal creates the reduced inner diameter within the valve lumen.

10. The catheter apparatus of claim 1, wherein the at least one protrusion is constructed out of a semi-compliant polymer so as to cooperate with the compression seal to form a seal therebetween when the compression seal creates the reduced inner diameter within the valve lumen.

11. The catheter apparatus of claim 1, wherein the at least one protrusion is constructed out of one or more radiopaque materials to facilitate positioning of the catheter within the valve lumen.

12. The catheter apparatus of claim 12, wherein the at least one protrusion is constructed out of a tungsten-loaded polymer.

13. The catheter apparatus of claim 12, wherein the at least one protrusion is constructed out of one or more of platinum, chromium, cobalt, tantalum, nitinol, gold, silver, bismuth subcarbonate, barium sulfate, bismuth oxychloride, bismuth trioxide, stainless steel or alloys thereof.

14. The catheter apparatus of claim 1, wherein the at least one protrusion has an outer diameter of about 100.1% to about 500% of the outer diameter of the catheter.

15. The catheter apparatus of claim 1, wherein the at least one protrusion has a length of about 0.001 inch to about 20 inches.

16. The catheter apparatus of claim 1, wherein the position of the at least one protrusion relative to the first end of the catheter indicates a corresponding distance by which the opposing second end of the catheter extends from the terminal end of the sheath when said protrusion is positioned substantially in line with the compression seal.

17. The catheter apparatus of claim 1, further comprising a plurality of spaced apart protrusions along a length of the catheter, each of the protrusions positioned relative to the first end of the catheter so as to indicate a corresponding distance by which the opposing second end of the catheter extends from the terminal end of the sheath when said protrusion is positioned substantially in line with the compression seal.

18. The catheter apparatus of claim 17, wherein the protrusions are spaced about 0.001 inch to about 20 inches apart from one another.

19. A catheter apparatus for use with a hemostasis valve, the hemostasis valve providing a valve body having a first end and an opposing second end with a valve lumen extending therebetween, the hemostasis valve further providing an elongate sheath engaged with the second end, and a compression seal positioned substantially proximal to the proximal end and configured for temporarily creating a reduced inner diameter within the valve lumen, the apparatus comprising:
an elongate catheter having a first end and an opposing second end;
an outer surface of the catheter having an outer diameter that is less than a neutral inner diameter of the valve lumen, thereby allowing the catheter to be slidably inserted a distance into the valve lumen;
the catheter having a length that is greater than a length of the sheath, such that the second end of the catheter may extend a distance beyond a terminal end of the sheath when the catheter is inserted a distance into the valve lumen; and
an at least one protrusion radially extending outwardly from the outer surface of the catheter and having an outer diameter that is less than the neutral inner diameter of the valve lumen but greater than the reduced inner diameter of the valve lumen, the at least one protrusion positioned relative to the first end of the catheter so as to indicate a corresponding distance by which the opposing second end of the catheter extends from the terminal end of the sheath when said protrusion is positioned substantially in line with the compression seal;
whereby, with the catheter positioned a distance within the valve lumen and the compression seal subsequently creating the reduced inner diameter within the valve lumen, the at least one protrusion contacts the compression seal and prevents the catheter from sliding toward the first end of the valve body.

20. A combination catheter and hemostasis valve system comprising:
a hemostasis valve comprising:
a valve body having a first end and an opposing second end with a valve lumen extending therebetween, the valve lumen having a neutral inner diameter;
an elongate sheath engaged with the second end of the valve body and in fluid communication with the valve lumen; and
a compression seal positioned substantially proximal to the first end of the valve body and configured for temporarily creating a reduced inner diameter within the valve lumen;
the hemostasis valve configured for selectively moving between one of a closed state—wherein the compression seal radially collapses so as to create the reduced inner diameter within the valve lumen—and an open state—wherein the compression seal radially moves outwardly so as to no longer create the reduced inner diameter within the valve lumen; and
a catheter apparatus comprising:
an elongate catheter having a first end and an opposing second end, an outer surface of the catheter having an outer diameter that is less than the neutral inner diameter of the valve lumen, thereby allowing the catheter to be slidably inserted a distance into the valve lumen; and
an at least one protrusion radially extending outwardly from the outer surface of the catheter, the at least one protrusion having an outer diameter that is less than the neutral inner diameter of the valve lumen but greater than the reduced inner diameter of the valve lumen;
whereby, with the catheter positioned a distance within the valve lumen and the hemostasis valve subsequently moved into the closed state, the at least one protrusion contacts the compression seal and prevents the catheter from sliding toward the first end of the valve body.
